(19) United States
(12) Patent Application Publication
(10) Pub. No.: US 2002/0133139 A1
(43) Pub. Date: Sep. 19, 2002

(54) Liquid cautery catheter

(76) Inventor: Harry Moulis, Ormond Beach, FL (US)

Correspondence Address: VAN DYKE & ASSOCIATES, P.A., 1630 HILLCREST STREET, ORLANDO, FL, 32803, US

(21) Appl. No.: 09/808,368

(22) Filed: Mar. 14, 2001

(51) Int. Cl. A61M 31/00

(52) U.S. Cl. 604/508; 604/523

(57) ABSTRACT

Disclosed herein are novel cautery devices, and methods and kits implementing the same. The disclosed devices are especially useful for localized delivery of a liquid caustic agent to treat various defects, malformations and injuries resulting in bleeding. The disclosed devices have uses in a number of medical disciplines, and specific examples are provided pertaining to treatment of defects, malformations, and injuries, or bleeding due to medical procedures, in and along the gastrointestinal tract.

### Background of the Invention

There are a number of vascular malformations, defects, or injuries that commonly occur along the lining of the intestine and other parts of the gastrointestinal tract. Some of the more common types include angiodysplasias or telangiectasias (esophageal, gastric, duodenal, jejunal, ileal, colonic, rectal; Helmrich et al., Southern Medical Journal 83:1450-1453 (1990)), watermelon stomach (Gretz and Achem, Am. J Gastroentero. 93:890-895 (1998); Binmoeller and Lieberman, Gastrointest Endosc 37:192-193 1991);, gastric antral vascular ectasias, and radiation injury (radiation proctitis, esophagitis, gastritis, enteritis). A typical characteristic of these types of disorders is undesired bleeding (Lewis, Gastroenterology Clinics of North America 23:67-91; and Jaspersen et al., Gastrointest Endosc 40:40-44 (1994)). Indeed, gastrointestinal bleeding accounts for at least 2% of all hospital admissions each year (Levy, N. Engl. J Med 290:1158 (1974)).

Conventional treatment of the foregoing disorders includes thermal treatment (Jensen et al. Gastointest Endosc 45:20-25 (1997); Askin and Lewis, Gastrointest Endosc 43:580-583 (1996), Argon Plasma coagulation ( Wahab et al., Endoscopy 29:176-181 (1997), and/or laser treatment (Taylor et aL, Gastrointest Endosc 52:353:357 (2000)). However, these conventional methods are not without their drawbacks. The medical equipment is relatively costly and can be cumbersome to use. Furthermore, they present the potential risks of perforation (Pierzchajilo, Colonoscopy, 22:451-470 (1995); Bedford et al., Am J Gastroenterol., 87:244-247 (1987)), or in the case of thermal treatment, heart disrythmias or even colonic explosions (Monahan et al, Gastrointest Endosc 38:40-43 (1992); Vellar et al., Br. J Surg. 73:157-158 (1986); Donato and Memeo, Dis Colon Rectum 36:291-292 (1993); Shinagawa et aL, Br. J Surg. 72:306 (1985)). Argon plasma coagulation has been shown to cause inflammatory polyps (Schmeck-Lindenau and Heine, Endoscopy 30:93-94 (1998).

U.S. Pat. Nos. 6,187,346 and 6,165,492 to Neuwirth et al. disclose chemical cauterization devices and methods used for treatment of lesions occuring in the uterus. The system taught in these patents involves filling the uterus with a caustic agent, such as silver nitrate, and then neutralizing the cauterizing agent with a sodium chloride solution. However, the methods taught in U.S. Pat. Nos. 6,187,346 and 6,165,492 are not applicable to situations where filling a cavity, such as the uterine cavity, is not possible. Furthermore, these patents do not teach devices that control delivery of a caustic agent as to allow for focal treatment of a confined area of tissue.

In view of the problems associated with traditional treatments, there is a need in the art for a cautery method that overcomes these problems, and provides an easy to use, inexpensive system for cauterization. While gastroenterologists encounter a number of chronic bleeding disorders, other medical disciplines, such as otorhinolaryngology, pulmonology, gynecology, urology, general surgery, thoracic surgery, and orthopedic surgery, may encounter deformations, defects, and/or injuries that result in undesired bleeding as well. Ideally, the new cautery method would be readily adaptable for use in medical procedures in the GI tract but also other organ systems.

### SUMMARY OF THE INVENTION

The subject invention is directed to a novel cautery system which provides localized cauterization and is easily adaptable for implementation in a number of surgical and non-surgical procedures. Specifically exemplified is a cautery system that delivers a liquid caustic agent to a site of need, wherein the liquid caustic agent is administered through the use of a catheter or other similar device. According to one aspect, the subject invention pertains to a catheter that has a regulating tip at one end, wherein the regulating tip has an impeder, such as, e.g., a sponge, fritted glass or other porous material disposed therein. As the tip contacts, or is placed proximate to, a site of need, a controlled amount of the caustic amount is released. Alternatively, the regulating tip has other configurations to allow for the controlled delivery of the caustic agent, such as the provision of a barrier having one or more small holes. The regulating tip enables controlled, focal delivery of the caustic agent whereby contact with non-target areas is avoided.

At the other end of the catheter, opposite to the regulating tip, the catheter is connected to a container for storing and supplying the caustic agent. A preferred container is a syringe comprising a plunger, barrel and a connecting end. More preferably, the catheter is equipped with an attachment means such as a female or male luer-lok end, which readily attaches to a syringe comprising the caustic agent.

According to a further aspect, the subject invention pertains to a method of delivering a caustic agent utilizing the cautery device of the subject invention. The subject method can be used to treat various malformations, defects, and injuries.

In yet another aspect, the subject invention pertains to a kit comprising a syringe, a catheter and a volume of a caustic agent.

These and other advantageous aspects of the subject invention will be described in further detail below.

### DESCRIPTION OF THE DRAWINGS

FIG. 1 shows an embodiment of the subject invention that comprises a catheter connected to a syringe containing a caustic agent.

FIG. 2 shows a magnified view of the tip of the catheter shown in FIG. 1.

FIG. 3 shows a number of alternate configurations of the regulating tip of the subject invention.

### DESCRIPTION OF THE PREFERRED EMBODIMENTS

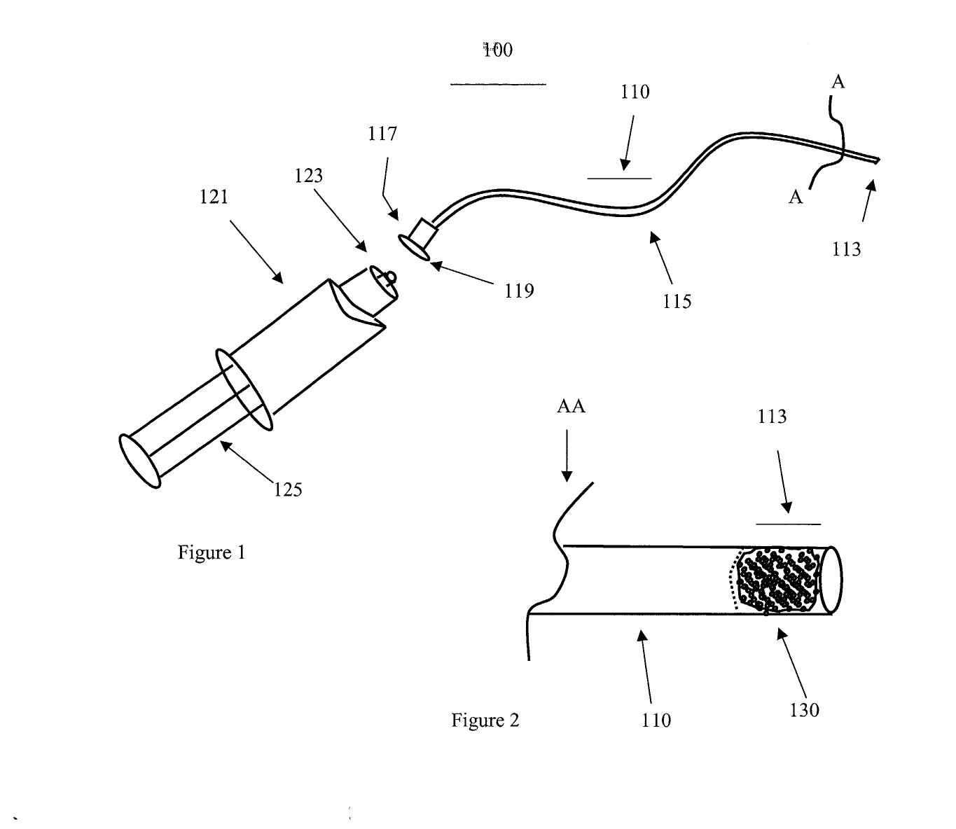

As discussed above, the subject invention is directed to medical devices useful as a cautery, and specifically for delivering a caustic agent to a site of need. Turning to the drawings, FIG. 1 shows an embodiment of the subject cautery device 100 that comprises a flexible catheter 110. The catheter 110 has a first end 113 (out of which a caustic agent is delivered), a flexible, elongated portion 115, and second end 117 that has a female luer-lock 119 connector disposed thereon for attaching to a male luer-lock end 123 of a syringe 121. During typical use of the cautery device 100, the syringe 121 is provided with an amount of a caustic agent and attached to the female luer-lock connector 119. The first end 113 and elongated portion 115 of the catheter 110 are guided through an endoscope and positioned proximate to site of need. By applying pressure to the plunger 125 of the syringe 121, the caustic agent travels through the catheter 110 and is ejected out at the first end 113 and onto the site of need in a controlled manner.

Shown in FIG. 2 is close-up depiction of the first end 113 of the catheter 110 up from the break line AA. FIG. 2 illustrates the placement of a permeable material 130 (fritted glass, sponge, etc.) in the first end 113, which governs the delivery of the caustic agent out of the catheter 110. The provision of the liquid permeable material 130 prevents uncontrolled spilling and flow of the caustic agent out of the first end 113, thereby limiting contact of the caustic agent with surrounding tissues. The permeable material 130 is preferably a sponge, fritted glass, or a semi-permeable membrane. Those skilled in the art, in view of the teachings herein, will readily appreciate that various materials can be used to make the permeable material.

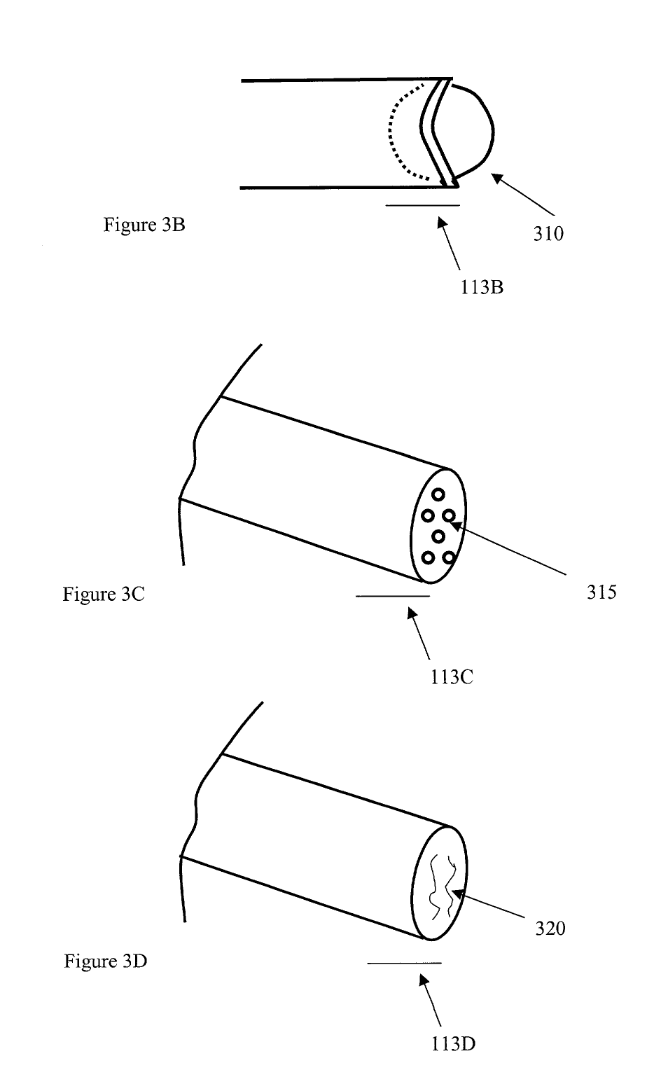

FIG. 3 shows alternative embodiments of the first end of the catheter which allows for controlled delivery of the caustic agent. FIG. 3A shows an embodiment which comprises a roll-on ball 310 attached to the first end 113B for applying caustic agent to the site of need. FIG. 3B shows an embodiment which comprises a closed first end 113C with a plurality of perforations 315 out of which caustic agent is ejected. FIG. 3C shows an embodiment which has a permeable membrane 320 rigidly attached to the first end 113D.

A number of conventional materials commonly used in the medical industry can be used to make the catheter 110. Examples of such materials include, but are not limited to, polyvinyl chloride, polyethylene, polypropylene, polyethylene terephthalate, polyurethane, polytetrafluoroethylene, fluoroethylenepropylene, or nylon, or combinations thereof. Examples of suitable materials are disclosed, e.g., in U.S. Pat. Nos. 6,165,166; 4,707,389, 3,561,493. The structural properties of the subject cautery device and catheter will be dictated by the intended use. For example, use of the subject cautery device with a flexible endoscope will require that the catheter is also flexible. Those skilled in the art will readily recognize appropriate materials for making such catheters to meet this requirement, as well as in the case where there is a need for a more rigid catheter.

The subject cautery device has a number of applications, in a number of different medical disciplines. With respect to gastroenterology, the subject invention may be useful to treat, for example, vascular malformations, watermelon stomach, gastric antral vascular ectasias, radiation injury, benign neoplasms, post-polypectomy bleeding, post-endoscopic ampullary sphincterotomy bleeding, ulcers, Dieulafoy's lesions, malignant neoplasms, Barrett's esophagus with or without dysplasia, varices, bleeding Mallory-Weiss tears, bleeding from portal hypertensive gastropathy, fistulae, or bleeding from colitis.

Examples of caustic agents appropriate for use with the teachings herein include, but are not limited to, silver nitrate, zinc chloride, copper sulfate, phenol, acids, alkali, iodine, potassium permanganate, or combinations thereof. Furthermore, depending on the intended use, the viscosity and strength or concentration of the selected caustic agent is routinely adjusted. Where deeper penetration of the caustic agent is preferred, a more concentrated solution of the caustic agent should be used. Other characteristics such as speed and severity of cautery are adjusted as well, depending on the desired use and may be achieved by altering viscosity.

The activity of the caustic agent is readily controlled by using silver compounds such as silver nitrate and silver thiocyanate or other compounds which can release silver ions. The silver ions react with the sulfides, proteins, and chlorides in cells. Since the sulfides and chlorides are vital to cell metabolism, the reaction results in necrosis of the cells. Another potentially useful agent is iodine which is radiopaque like silver. Compositions containing iodine react with the target tissue as the result of the release of elemental free Iodine and the reaction can be stopped by forming a stable compound, for example, sodium iodide. In an especially preferred embodiment, silver nitrate and DEXTRAN 70® are utilized together because they are easy to work with, are controllable, and are recognized by the medical profession and government regulatory agencies as acceptable agents for human use. DEXTRAN 40®. and 70® can be used intravenously and intramuscularly and in several organ systems such as the genital tract. Silver Nitrate is used on the skin, upper respiratory tract, lower genital tract, and other locations. The silver ion has a loose but stable binding with the dextran carrier but is pulled off by the consumption of the ion at the tissue sites by binding to anions and protein. The carrier may be made of dextrans or glucose or other sugars used in intravenous solutions but preferably in concentrations sufficient to form gels or pastes. The compositions prepared in accordance with this invention have a viscosity that is suitable for their intended purpose at temperatures between about 20° C. and about 37° C., however, the viscosity may be adjusted as specific applications dictate. Alginates, aloe, carboxymethylcellulose, silicones and oxidized cellulose may also be used to form pastes and gels but the dextrans and sugars are the preferred choices because of their acceptance by the medical profession and regulatory agencies.

The speed and severity of the chemical necrosis may be regulated by the percentage of the silver nitrate in the paste. By increasing the percentage of the silver nitrate in the paste the possibility for a deeper burn is increased. It is possible, by procedures well known to those skilled in the art, to determine the appropriate concentration of silver nitrate to achieve the desired depth of cauterization for specific applications. The practitioner may readily formulate a paste that is essentially self regulating. For example, a weak silver nitrate paste may be formulated that will expend itself after necrosing to a depth of only half the maximum safely allowable depth, thereby reducing the danger of necrosing too deeply. Preferably, the composition comprises 1-50%, by weight, of caustic agent. More preferred, the caustic agent comprises 10-40%, by weight of the composition. Alternatively, the practitioner may easily terminate the treatment by introducing a normal saline solution, e.g., NaCl, which will deactivate the silver nitrate by forming silver chloride. An advantage of the silver nitrate is that the deactivating agent for the silver ion is the chloride ion found in several solutions used regularly in medicine, e.g., intravenously and intramuscularly, such as normal saline or Ringer's solution. The silver nitrate deactivation is the essentially stoichiometric formation of an insoluble non-caustic precipitate.

The viscosity of the caustic composition may be adjusted so that it does not flow uncontrollably from the site of need. The caustic composition should flow easily, i.e, without excessive pressure, through a catheter having an inside diameter of about 2 mm. Preferably, the caustic composition should be thick enough that it does not run, i.e, it stays in the vicinity of the point of application. In a preferred embodiment, a caustic composition having a consistency ranging from toothpaste to pancake syrup is utilized as specific applications dictate. Thixotropic caustic compositions utilizing, e.g., mineral clays or the like, may be especially useful in some applications. While modifying the viscosity of the cauterizing compound can alter the flow properties and therefore aid in the control of delivery, the subject cautery device allows for controlled delivery of a cauterizing agent having a broad range of viscosities as result of its regulating tip.

Preferably, about 10 gms of DEXTRAN 70® is mixed with about 10 ml of water containing varying concentrations of silver nitrate, which flows slowly and smoothly under pressure through a 2 mm catheter 30 cm to 260 cm long attached to a 10 ml syringe. However, the concentrations may be varied as specific applications dictate to meet the conditions of delivery and the organ or structure to be treated. The low viscosity or “watery” compositions comprise about 5 gms of DEXTRAN 70® and about 10 ml water and the high viscosity or “thick paste” compositions are comprised of about 10 gms to about 15 gms or higher of DEXTRAN 70® and about 10 ml water.

The teachings of all patents and publications cited throughout this specification are incorporated by reference in their entirety to the extent not inconsistent with the teachings herein.

It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A cautery device comprising:
a catheter comprising a first end and a second end, wherein said first end comprises a regulating tip to govern delivery of a caustic agent from said catheter to a site of need; and
a container for supplying said caustic agent to said catheter attached to said second end.

2. The cautery device of claim 1, wherein said regulating tip comprises a liquid permeable material positioned therein to regulate flow of said caustic agent.

3. The cautery device of claim 2, wherein said liquid permeable material comprises a sponge, fritted glass, semi-permeable membrane, or combinations thereof.

4. The cautery device of claim 1, wherein said regulating tip comprises a closed end having one or more holes defined thereon.

5. The cautery device of claim 1, wherein said catheter is flexible.

6. The cautery device of claim 1, wherein said container comprises a syringe or pump.

7. The cautery device of claim 1 wherein said caustic agent comprises silver nitrate, zinc chloride, copper sulfate, phenol, acids, alkali, iodine, potassium permanganate, or combinations thereof.

8. The cautery device of claim 1 wherein said catheter is comprised of polyvinyl chloride, polypropylene, polyethylene, polyethylene terephthalate, polyurethane, polytetrafluoroethylene, fluoroethylenepropylene or nylon, or combinations thereof.

9. The cautery device of claim 1 wherein said regulating tip comprises an impeder which governs the flow of delivery of said caustic agent to the site of need as to limit undesired spillage of said caustic agent than without said impeder, thereby avoiding damage to tissues surrounding the site of need.

10. A method of administering a caustic agent to a site of need comprising the steps of:
obtaining a cautery device comprising a catheter that comprises a first end and a second end, wherein said first end comprises a regulating tip to govern delivery of a caustic agent from said catheter to a site of need; and a container for supplying said caustic agent to said catheter attached to said second end;
providing a caustic agent in said container; and
delivering said caustic agent to said site of need.

11. The method of claim 10, wherein said caustic agent is silver nitrate, zinc chloride, copper sulfate, phenol, acids, alkali, iodine, potassium permanganate, or combinations thereof.

12. The method of claim 10, wherein said container is a syringe or pump.

13. The method of claim 10, wherein said site of need is located along a recipient's gastrointestinal tract.

14. The method of claim 10, wherein said regulating tip comprises an impeder which governs the flow of delivery of said caustic agent to the site of need as to limit undesired spillage of said caustic agent than without said impeder, thereby avoiding damage to tissues surrounding the site of need.

15. The method of claim 10, wherein said method is used to treat vascular malformations, watermelon stomach, gastric antral vascular ectasias, radiation injury, benign neoplasms, post-polypectomy bleeding, post-endoscopic ampullary sphincterotomy bleeding, ulcers, Dieulafoy's lesions, malignantneoplasms, Barrett's esophagus, varices, bleeding Mallory-Weiss tears, bleeding from portal hypertensive gastropathy, fistulae, or bleeding from colitis.

16. A kit comprising a catheter, wherein said catheter comprises a first end and a second end, and wherein said first end comprises a regulating tip to govern delivery of a caustic agent from said catheter to a site of need; a syringe; and a volume of a liquid caustic agent.

17. The kit of claim 16, wherein said regulating tip comprises an impeder which governs the flow of delivery of said caustic agent to the site of need as to limit undesired spillage of said caustic agent than without said impeder, thereby avoiding damage to tissues surrounding the site of need.

18. An article of manufacture comprising a catheter, wherein said catheter comprises a first end and a second end, and wherein said first end comprises a regulating tip to govern delivery of a caustic agent from said catheter to a site of need; a syringe; a volume of a liquid caustic agent; and packaging materials; wherein said regulating tip comprises an impeder which governs the flow of delivery of said caustic agent to the site of need as to limit undesired spillage of said caustic agent than without said impeder, thereby avoiding damage to tissues surrounding the site of need.

19. A kit comprising a catheter, wherein said catheter comprises a first end and a second end, and wherein said first end comprises a regulating tip to govern delivery of a caustic agent from said catheter to a site of need; and a syringe; wherein said regulating tip comprises an impeder which governs the flow of delivery of said caustic agent to the site of need as to limit undesired spillage of said caustic agent than without said impeder, thereby avoiding damage to tissues surrounding the site of need.
